(19) United States
(12) Patent Application Publication
(10) Pub. No.: US 2007/0006887 A1
(43) Pub. Date: Jan. 11, 2007

(54) Tracking system for prosthetic and implantable devices

(75) Inventor: Milton Frank, Bergenfield, NJ (US)

Correspondence Address: EPSTEIN DRANGEL BAZERMAN & JAMES, LLP, 60 EAST 42ND STREET, SUITE 820, NEW YORK, NY, 10165, US

(73) Assignee: Med-Track Partners LLC

(21) Appl. No.: 11/482,780

(22) Filed: Jul. 6, 2006

Related U.S. Application Data
(60) Provisional application No. 60/697,874, filed on Jul. 8, 2005.

(51) Int. Cl. A61B 19/00

(52) U.S. Cl. 128/899; 340/286.01; 340/146.2; 340/999

(57) ABSTRACT

A system is provided for tracking prosthetic and implantable devices. The system includes, in combination, a prosthetic or implantable device and an RFID transducer fixed to the device and having lot identification information stored therein. A wand is provided which is capable of scanning the transducer and reading the recorded information. Means are also provided for storing the read information.

### BACKGROUND OF THE INVENTION

1. Field of the Invention

The present invention relates to prosthetic and other types of implantable devices and more particularly to a method and apparatus for tracking such devices.

2. Description of Prior Art Including Information Disclosed Under 37 CFR 1.97 and 1.98

Prosthetics are commonly used in medicine to replace parts of the body lost as a result of injury, wear, disease, or trauma, or parts missing from birth. Pacemakers and defibrillators are commonly implanted and recalled. These devices are extremely expensive and tracking the use and supply levels of these devices is beneficial. An RFID tracking system for such devices would be very beneficial.

Recalls in the prosthesis industry are common, and as such, the availability of a prosthesis tracking system is necessary to identify and then replace recalled prostheses. For example, Exactech, Inc., of Gainesville, Fla., had to recall its Cemented Trapezoid Tibial Tray Sz. 4F/4T, Knee joint patellofemorotibial polymer/meta V polymer semi-constrained cemented prosthesis because the implant was identified incorrectly at the time of manufacture and was then labeled and distributed with the incorrect size information. Thus, a Cemented Trapezoid Tibial Tray labeled as 4F/4T appeared to dimensionally be a 3F/3T.

Nobel Biocare USA, Inc., Yorba Linda, Calif. recalled a Replace 4.3 mm Conical Abutment, 4.3 mm diameter×4.5 mm height, Catalog #43503. Such an abutment is used either for single or multiple units, screw-retained prosthetic restorations. It was recalled because the conical abutment was not the size as labeled. Nobel Biocare USA, Inc. also had to recall its IMC (Intramobile Connector) Conversion Screw, 3.5 mm diameter, Catalog #8198CA, because the screw in the package was incorrect. An IMC cylinder is used for single or multiple units, screw-retained prosthetic restorations.

Advanced technology has brought significant improvements to many types of prosthetic devices. Lower extremity trauma amputees, on average, use a new prosthesis every two years. See: Use and Satisfaction with Prosthetic Devices Among Persons with Trauma-Related Amputations: A Long-Term Outcome Study. American Journal of Physical Medicine & Rehabilitation. 80(8):563-571, August 2001. Dillingham, Timothy R. MD, MS; Pezzin, Liliana E. PhD; MacKenzie, Ellen J PhD; Burgess, Andrew R. MD

A tracking system with device history records would ease the process of replacing outdated devices. Prosthetics suppliers could be aware of the sale of a given prosthesis immediately. This would allow the supplier to bill the customer sooner and then restock them with another device. A scan of the prosthetic device could also identify a person who is unconscious or deceased, and it could reveal other aspects of a patient's medical history which would be needed for various types of treatments. This has both medical and forensic benefits. A tracking system could also reduce occurrences of infection in prostheses by providing superior control of device expiration/shelf-life.

Hospital inventory control could be greatly enhanced by a tracking system. Such a system could provide for easy verification that correct components are being used. Further, long term data on implant age could easily be obtained.

However, to the best of my knowledge, no such tracking system is currently available which is capable of performing these functions.

It is, therefore a prime object of the present invention to provide a tracking system for prosthetic and other types of implantable devices.

It is another object of the present invention to provide a tracking system for prosthetic and other types of implantable devices capable of tracking lot numbers on such devices.

It is another object of the present invention to provide a tracking system for prosthetic and other types of implantable devices that uses an RFID transducer operating in the frequency range of 50 kHz to 1 MHz.

It is another object of the present invention to provide a tracking system for prosthetic and other types of implantable devices wherein the RFID transducer is situated internally in a non-metallic part of the device.

It is another object of the present invention to provide a tracking system for prosthetic and other types of implantable devices wherein the RFID transducer is situated just below the surface in a metallic part of the device.

It is another object of the present invention to provide a tracking system for prosthetic and other types of implantable devices wherein the transducer is bonded on the device using medical grade epoxy.

### BRIEF SUMMARY OF THE INVENTION

In accordance with the present invention, apparatus is provided for tracking prosthetic and implantable devices. The apparatus includes, in combination, a prosthetic or implantable device and an RFID transducer fixed to the device and having lot identification information stored therein. A wand is provided which capable of scanning the transducer and reading the recorded information. Means are provided for storing the read information.

The transducer preferably operates at a frequency range of 50 KHZ to 1 MHZ.

The device may include a non-metallic part. In that case, the transducer is situated internally in the non-metallic part.

The device may include a metallic part. In that case, the transducer is situated proximate the surface of the metallic part.

The apparatus further includes means for fixing the transducer on the device. The fixing means preferably takes the form of medical grade epoxy.

The device may consist of multiple components. In that case, an RFID transducer is fixed to each of the components. Each of the transducers is preferably located in a specific quadrant of the component.

The apparatus may be used in a warehouse. In that case, the device is scanned with the wand prior to being shipped for medical use.

The apparatus may be used in a medical facility. In that case, the device is scanned with the wand as it is implanted into the patient or when the patient returns for further treatment.

The storing means may include information relating to the device history or comprises information relating to the patient's medical history.

The apparatus may also include means for transmitting the read information to the device vendor.

If multiple components are involved, means may be provided for verifying that the correct components of the device have been selected for use.

The apparatus may further include means for recording the medical history of the patent in the transducer.

### BRIEF DESCRIPTION OF THE SEVERAL VIEWS OF DRAWINGS

To these and to such other objects that may hereinafter appears, the present invention relates to a system for tracking prosthetic and implantable devices as described in detail in the following specification and recited in the annexed claims, taken together with the accompanying drawings, in which like numerals refer to like parts and in which:

FIG. 1 is an isometric view of a knee tibia implant made of non-metallic material with an RFID transducer implanted therein and a wand for reading the information from the transducer;

FIG. 2 is an isometric view of a two-part metallic knee femer implant showing RFID transducers situated near the surface in each part; and

FIG. 3 is a block diagram showing the electronic components of the system.

### DETAILED DESCRIPTION OF THE INVENTION

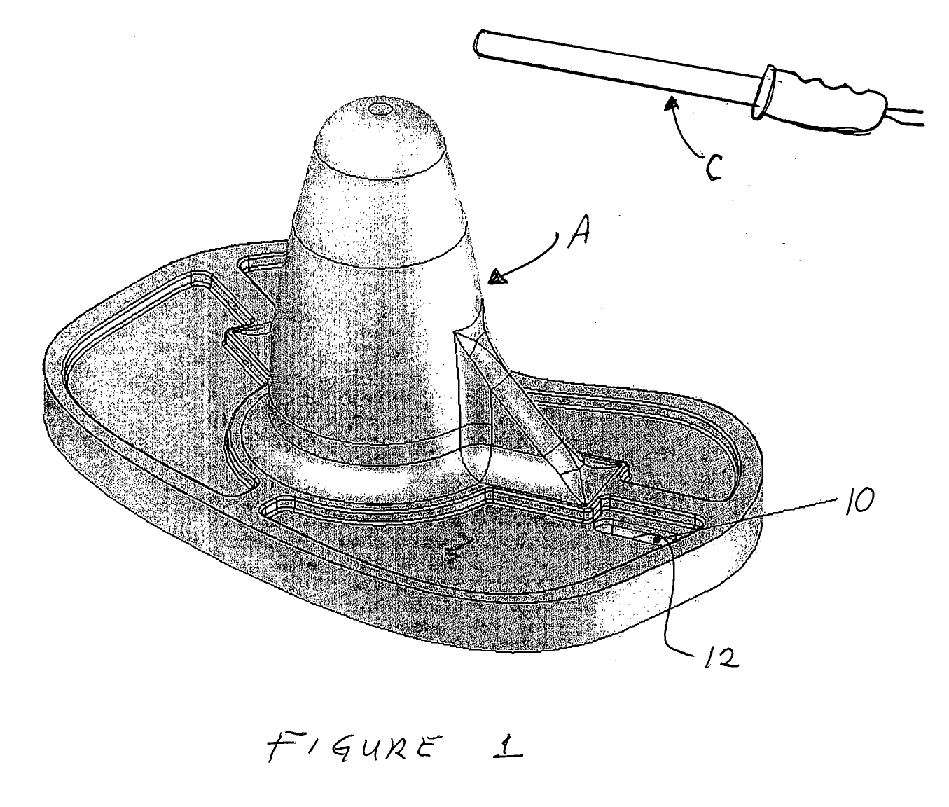

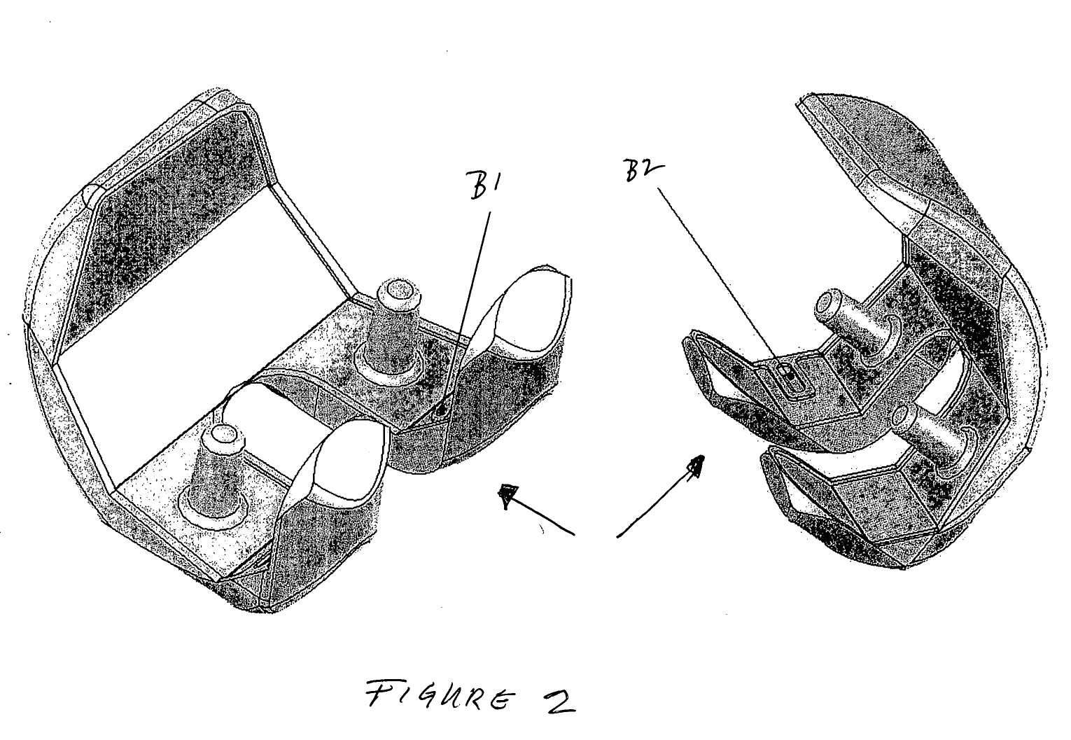

The apparatus of the present invention is provided for tracking prosthetic and implantable devices, for example the single-part artificial knee tibia, generally designated A, as seen in FIG. 1 or the two-part knee femer, generally designated B, as seen in FIG. 2. In combination with the prosthetic or implantable device, an RFID transducer 10 fixed to the device, and in particular, within a recess 12 formed in the device for that purpose. Transducer 10 contains lot identification information stored therein. However, other information may be stored in transducer 10, as well. For example, the transducer may contain information as to the identity of the patient or the medical history of the patient.

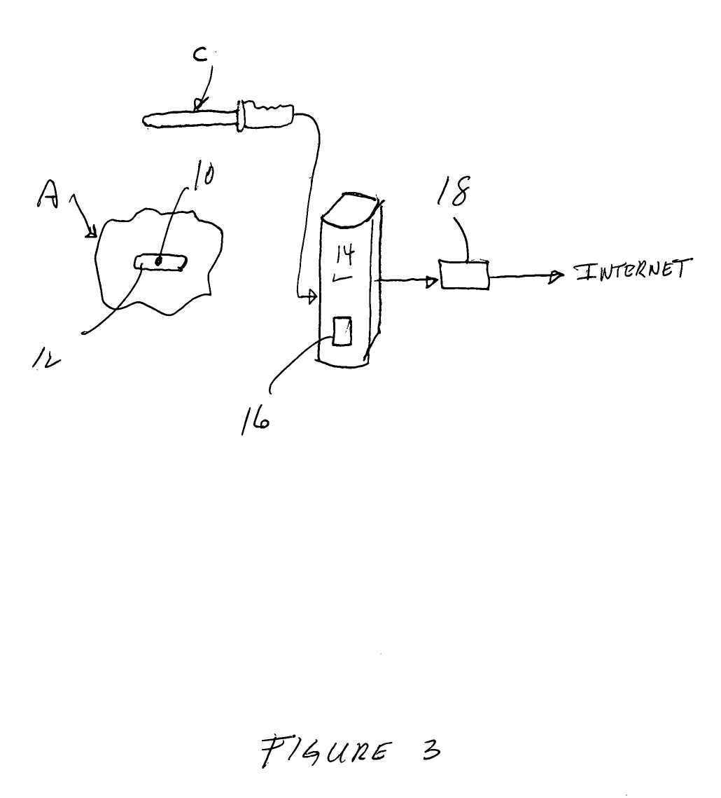

A wand, generally designated C, is provided, as illustrated in FIG. 1. Wand C capable of scanning transducer 10 and reading the information recorded therein. As illustrated in FIG. 3, the read information scanned by the wand is forwarded to a computer 14 which includes memory means 16 for storing the read information.

Transducer 10 preferably operates at a frequency range of 50 KHZ to 1 MHZ.

The device A may include a non-metallic part. In that case, the transducer is situated internally in the non-metallic part.

The device B may include a metallic part. In that case, the transducer is situated proximate the surface of the metallic part.

Means are provided for fixing the transducer on the device. The fixing means preferably takes the form of medical grade epoxy.

The device B may consist of multiple components, B1 and B2, as shown in FIG. 2. In that case, an RFID transducer 10 is fixed to each of the components. Each of the transducers 10 is preferably located in a specific quadrant of the component.

The apparatus may be used in a warehouse. In that case, the device is scanned with the wand prior to being shipped for medical use to obtain and store the identification information. The apparatus may be used in a medical facility. In that case, the device is scanned with the wand as it is implanted into the patient or when the patient returns for further treatment.

The memory means 16 may store information relating to the device history or information relating to the patient's medical history, as well as the identification information. In the latter case, the RFID enabled device allows the patient to have electronically available vital medical data.

The apparatus may also include means for transmitting the read information to the device vendor. That can simply be an Internet connection between computer 14 and the vendor's computer using a modem 18.

If multiple components are involved, computer 16 may contain software for verifying that the correct components of the device have been selected for use.

The apparatus may further include means for recording the medical history of the patent in the transducer.

During a replacement procedure the physician can determine which tools will be required to replace the implant by being able to identify what was previously implanted. The physician will be able to use the information obtained from the transducer to choose the proper replacement component.

In the operating room the physician can have the computer verify that the implants are the ones that were ordered for the patient.

In the physician's office, the apparatus can be used to verify the type and size of implant in the patient. Thus, in the event of a recall, the physician can verify non-invasively whether the implant in a specific patient needs to be replaced.

It will now be appreciated that the present invention relates to a system for tracking prosthetic and implantable devices. The system includes, in combination, a prosthetic or implantable device and an RFID transducer fixed to the device and having lot identification information stored therein. A wand is provided which is capable of scanning the transducer and reading the recorded information. Means are also provided for storing the read information.

While only a limited number of preferred embodiments of the present invention have been disclosed for purposes of illustration, it is obvious that many modifications and variations could be made thereto. It is intended to cover all of those modifications and variations which fall within the scope of the present invention, as defined by the following claims.

I claim:

1. Apparatus for tracking prosthetic and implantable devices comprising, in combination, a prosthetic or implantable device and an RFID transducer fixed to said device and having a lot identification information recorded therein, a wand capable of scanning said transducer and reading said recorded information and means for storing said read information.

2. The combination of claim 1 wherein said transducer operates at a frequency range of 50 KHZ to 1 MHZ.

3. The combination of claim 1 wherein said device comprises a non-metallic part and wherein said transducer is situated internally in said non-metallic part.

4. The combination of claim 1 wherein the device comprises a metallic part and said transducer is situated proximate the surface of said metallic part.

5. The combination of claim 1 further comprising means for fixing said transducer on the device.

6. The combination of claim 5 wherein said fixing means comprises medical grade epoxy.

7. The combination of claim 1 wherein said device comprises multiple components and further comprising an RFID transducer fixed to each of said components.

8. The combination of claim 7 wherein each of said transducers is located in a specific quadrant of the component.

9. The combination of claim 1 for use in a warehouse wherein said device is scanned with said wand prior to being shipped for medical use.

10. The combination of claim 1 for use in a medical facility wherein said device is scanned with said wand as it is implanted.

11. The combination of claim 1 for use in a medical facility wherein said device is scanned with said wand when a patient returns for further treatment.

12. The combination of claim 1 wherein said storage means further comprises information relating to the device history.

13. The combination of claim 1 wherein said storage means further comprises information relating to the patient's medical history.

14. The combination of claim 1 further comprising means for transmitting the stored information to the device vendor.

15. The combination of claim 7 further comprising means for verifying that the correct components of the device has been selected for use.

16. The combination of claim 1 further comprising means for recording the medical history of the patent in the transducer.
